(19) United States
(12) Patent Application Publication
(10) Pub. No.: US 2024/0024299 A1
(43) Pub. Date: Jan. 25, 2024

(54) USE OF DRUGS THAT STIFFEN MATURE GAMETOCYTES FOR BLOCKING TRANSMISSION OF PLASMODIUM PARASITES

(71) Applicants: INSERM (INSTITUT NATIONAL DE LA SANTÉ ET DE LA RECHERCHE MÉDICALE), Paris (FR); UNIVERSITÉ PARIS CITÉ, Paris (FR); ASSISTANCE PUBLIQUE - HÔPITAUX DE PARIS, Paris (FR); UNIVERSITE ANTILLES GUYANE, Guadeloupe (FR)

(72) Inventors: Pierre BUFFET, PARIS (FR); Papa Alioune NDOUR, Paris (FR); Mario CARUCCI, PARIS (FR); Julien DUEZ, EVRY-COURCOURONNES (FR)

(21) Appl. No.: 18/257,438

(22) PCT Filed: Dec. 13, 2021
(86) PCT No.: PCT/EP2021/085497

§ 371 Date: Jun. 14, 2023

(30) Foreign Application Priority Data
Dec. 14, 2020 (EP) 20306560.2

(51) Int. Cl. A61K 31/438; A61K 31/496; A61K 31/4745; A61K 45/06; A61P 33/06

(52) U.S. Cl. CPC A61K 31/438; A61K 31/496; A61K 31/4745; A61K 45/06; A61P 33/06

(57) ABSTRACT

The spleen clears rigid erythrocytes from the circulation. Drug-induced stiffening of Plasmodium falciparum intra-erythrocytic sexual stages (mature gametocytes) is therefore expected to block the transmission of malaria By screening 13 555 compounds with spleen-mimetic microfilters, the inventors identified 82 compounds that stiffen mature gametocytes. Eight active families were identified, including known anti-malarial, antimicrobial or anticancer agents, amongst others. Hit prioritization based on accessible safety and pharmacokinetics data in humans identified 3 leading candidates. NITD609 displayed killing and stiffening effects (IC50 of 100 and 50 nM, respectively), while TD-6450 and L-THP had a pure or predominant stiffening effect (IC50 of 600 and 5 nM, respectively). These values are lower than or close to peak plasma concentrations in humans. Clinical trials with these strong malaria transmission-blocking candidates are envisioned.

### FIELD OF THE INVENTION

The present invention is in the field of medicine, in particular parasitology.

### BACKGROUND OF THE INVENTION

Protozoan parasites of the genus Plasmodium cause diseases (malaria) in humans and in many animal species. In humans, Plasmodium falciparum is the most common cause of malaria and is responsible for about 80% of all malaria cases, and is also responsible for about 90% of the deaths from malaria in humans. Plasmodium falciparum initially infects the liver, but then moves into the blood, where it multiplies and persists through an asexual replication cycle in red blood cells (also known as RBCs, haematids or erythrocytes). The RBC is thus the main host cell for Plasmodium falciparum (asexual and sexual erythrocytic stage). The release and persistence in bloodstream are prerequisites for mature gametocytes to be taken up by mosquitoes and ensure parasite transmission. It has been shown that release into the blood circulation is concomitant with an increase in infected RBC deformability that allows mature gametocytes to circulate through the spleen (Tiburcio et al, Blood 2012). Accordingly, targeting mechanisms that regulate gametocyte deformability would promote clearance of mature gametocytes by the spleen and reduce their ability to circulate in peripheral blood, thereby opening novel avenues in the present strategies to reduce parasite transmission.

### SUMMARY OF THE INVENTION

The present invention is defined by the claims. In particular, the present relates to methods of blocking transmission of plasmodium parasites.

### DETAILED DESCRIPTION OF THE INVENTION

The spleen clears rigid erythrocytes from the circulation. Drug-induced stiffening of Plasmodium falciparum intra-erythrocytic sexual stages (mature gametocytes) is therefore expected to block the transmission of malaria. By screening 13 555 compounds with spleen-mimetic microfilters, the inventors identified 82 compounds that stiffen mature gametocytes. Eight active families were identified, including known anti-malarial, antimicrobial or anticancer agents, amongst others. Hit prioritization based on accessible safety and pharmacokinetics data in humans identified 3 leading candidates. NITD609 displayed killing and stiffening effects (IC50 of 100 and 50 nM, respectively), while TD-6450 and L-THP had a pure or predominant stiffening effect (IC50 of 600 and 5 nM, respectively). These values are lower than or close to peak plasma concentrations in humans. Clinical trials with these strong malaria transmission-blocking candidates are envisioned.

Thus, the present invention relates to a method of blocking transmission of a Plasmodium parasite by an infected subject comprising administering to the subject a therapeutically effective amount of a drug capable of increasing the rigidity of iRBCs selected from the group consisting of TD-6450, NITD609, and L-THP.

As used herein, the terms “subject” and “patient” are used interchangeably herein and will be understood to refer to warm blooded animals, for example, mammals and birds, particularly mammals. Non-limiting examples of animals within the scope and meaning of this term include dogs, cats, rats, mice, guinea pigs, chinchillas, horses, goats, cattle, sheep, zoo animals, Old and New World monkeys, non-human primates, and humans, and any other animal susceptible to malaria.

In some embodiments, the subject can be human or any other animal (e.g., birds and mammals) susceptible to infection by plasmodium parasites (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult.

In some embodiments, the subject is infected by a Plasmodium parasite selected from the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, and more preferably from the group consisting of Plasmodium falciparum, Plasmodium vivax, Plasmodium ovate, Plasmodium knowlesi and Plasmodium malariae, and more preferably from the group consisting of Plasmodium falciparum and Plasmodium vivax. In some embodiments, said parasite is Plasmodium falciparum, in particular the Palo Alto I strain of Plasmodium falciparum.

The method of the present invention is particularly suitable for inducing the spleen-dependent clearance of mature gametocytes-hosting RBCs from the circulating blood. In particular the drug of the present invention indeed makes gametocytes unavailable to Anopheles sp. (i.e. mosquitos) thereby removing them from the transmission cycle.

The method of the present invention is thus particularly suitable for blocking transmission of malaria.

As used herein, the term “iRBCs”, or “Plasmodium-infected-iRBCs”, it is meant herein ring-RBCs (or ring-hosting RBCs) and/or gametocytes-hosting RBCs, in particular mature gametocytes-hosting RBCs.

As used herein, the expression “capable of increasing rigidity of iRBCs”, it is meant herein that the drug is capable of increasing rigidity of iRBCs by at least 5%, preferably at least 10% and more preferably at least 15%. The ability of the drug to increase rigidity of iRBCs can be in particular assessed by measuring the deformability of iRBCs cultured in the presence and in the absence of said drug. Thus, as used herein the expression “increase rigidity” means “decrease deformability” and in particular “decrease deformability by at least 5%, preferably at least 10% and more preferably at least 15%”.

In some embodiments, the drug is TD-6450 (i.e. carbamic acid, N-[(1S)-1-[[(2S)-2-[5-[4′-[[[6-[(2R,5S)-2,5-dimethyl-4-[(methyl amino)carbonyl]-1-piperazinyl]-3-pyridinyl]carbonyl]amino]-2′-(trifluoromethoxy) [1,1′-biphenyl]-4-yl]-1H-imidazol-2-yl]-1-pyrrolidinyl]carbonyl]-2-methylpropyl]-, methyl ester) (CAS No.: 1374883-22-3) having the formula of:

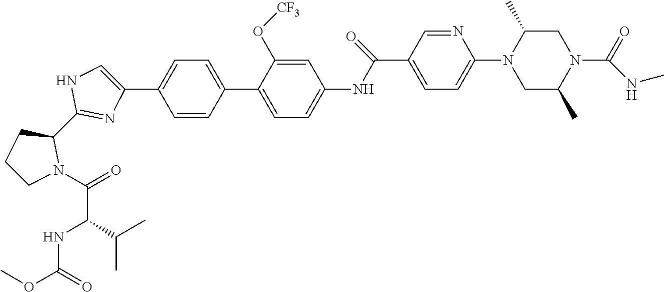

In some embodiments, the drug is Cipargamin (NITD609, KAE609) ((1R,3S)-5′,7-Dichloro-6-fluoro-3-methyl-spiro[2,3,4,9-tetrahydropyrido[3,4-b]indole-1,3′-indoline]-2′-one) (CAS No.:1193314-23-6) having the formula of:

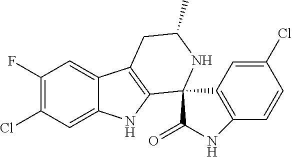 [SMILES: C[C@H]1Cc2c([nH]c3cc(Cl)c(F)cc23)[C@@]2(N1)C(=O)Nc1ccc(Cl)cc12]

In some embodiments, the dug is levo-tetrahydropalmatine (L-THP) ((13aS)-2,3,9,10-tetramethoxy-6,8,13,13a-tetrahydro-5H-isoquinolino[2,1-b]isoquinoline) (CAS No.: 483-14-7) having the formula of:

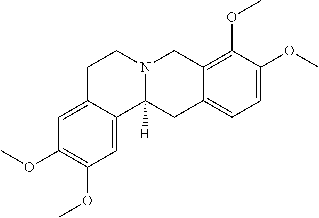

In some embodiments, the drug capable of increasing the rigidity of iRBCs is TD-6450 and is administrated in combination with NITD609.

As used herein, the term “in combination” encompasses simultaneous, separate, or sequential use of two therapeutic compounds.

In some embodiments, the method of the present invention further comprises administering to the mammal at least one additional antimalarial compound. Any suitable antimalarial compound can be used, many of which are well known in the art. Non-limiting examples of suitable antimalarial compounds include primaquine, bulaquine, artemisinin and derivatives thereof, chloroquine, hydroxychloroquine, mefloquine, amodiaquine, piperaquine, pyronaridine, atovaquone, tafenoquine, methylene blue, trioxaquines, endoperoxides such as OZ 439 and OZ 277, decoquinate, 9-anilinoacridines, doxycycline, azithromycine, erythromycine, spiramycine, pyrimethamine, sulfadiazine, sulfamethoxazole, HIV-protease inhibitors, and natural products such as neem, epoxomicin, harmonine, and riboflavin.

In some embodiments, the drug can be administered at the beginning or at the end of a conventional curative treatment of a malaria attack by a known anti-malarial agent, as primaquine is added to an artemisnin-based combination therapy for its transmission-blocking potential, as recommended by the WHO in areas of low transmission of malaria.

As used herein, the term “therapeutically effective amount” of the drug of the present invention is meant a sufficient amount of the compound to block the transmission of the Plasmodium parasite at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated, the potential positive impact of treatment for the local or general human community, and the severity of the disorder or impact of Plasmodium carriage by the subject on the transmission of malaria and health of the surrounding population; activity of the specific compound employed; the specific composition employed, the age, body weight, general health, sex and diet of the patient; the time of administration, route of administration, and rate of excretion of the specific compound employed; the duration of the treatment; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the drug of the present invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising the compound of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The compound of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to compound formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1. Screening progression cascade. ReFrame library (12,805 compounds), Kinase Inhibitors Box (350 compounds) and Pathogen Box (400 compounds) were screened with “microsphiltration” against P. falciparum gametocytes. Hits from primary screening were further selected by dose-response analysis (DRA). Confirmed 82 hits were divided in 7 groups, based on their activity and molecular target (data not shown). Hits repartition is shown in parenthesis, along with the name of one representative hit for each group. A score was assigned to each DRA confirmed hit, based on route of administration, safety in human clinical trials, pharmacokinetic and affordability for developing countries, leading to final selection of 3 hits for further in vitro and in vivo confirmation experiments.

FIG. 2. Representative single 384-wells plate scatterplots of primary screening. Each well of a screening plate is loaded with pre-spotted compounds while two columns are left for controls. They are represented as follows: empty circles for non-selected compounds and black circles for hits (352 wells), black squares for negative control (DMSO 0.05%, 16 wells), black squares for main positive control (NITD609 0.5 μM, 8 wells), light black empty squares for retention positive control (Calyculin A 75 nM, 6 wells), black squares for killing control (Gentian Violet 50 μM, 2 wells). For each well, killing and retention values are calculated and distributed in a “xy” graph. Two graphs are made for each plate: one using male and female gametocytes imaging (“readout 1”, left panel) and one using specific female gametocytes imaging (“readout 2”, right panel). For each graph, a linear regression (solid line) is tracked with 95% confidence interval (dashed lines). The compounds beyond the upper confidence interval in both readouts are identified as hits. A) Plate number 15 of ReFRAME library: 8 hits with readout 1, 11 hits with readout 2, 4 hits (shared between the two readouts) finally identified. B) Plate number 9 of ReFRAME library: 7 hits with readout 1, 11 hits with readout 2, 5 hits finally individuated, including NITD609 (highlighted with an arrow). In few cases, compounds with only killing effect were added to hitlist.

FIG. 3. Scatterplot representation of three different libraries screened. Non-selected compounds are depicted as empty circles, hits are highlighted in black. Calyculin (75 nM) results are depicted as empty black squares and NITD609 (0.5 μM) as black squares. X axis corresponds to killing rates and Y axis corresponds to retention rates normalized on negative control values in the same plate (average of two readouts in both killing and normalized retention values). A) Kinase Inhibitors Box, 350 compounds (average of six repetitions) and Pathogen Box, 400 compounds (average of three repetitions) libraries. Hits and controls are labelled with a “K” if referred to Kinase Inhibitors Box and with a “P” if referred to Pathogen Box. B) ReFRAME library, 12.8 k compounds. L-THP is not present in this graph as deficient well during primary screening.

FIG. 4. Examples of dose-response curves (DRCs) of confirmed hits. Every hit from primary screening has been re-tested in duplicate (ReFrame library) and in triplicate (Kinase Inhibitors and Pathogen Box libraries) for dose-response, starting from 5 (10 μM for ReFrame hits) to 5 nM. For each of them, a DRC for killing and retention was tracked and IC₅₀ was calculated (one for readout 1, male and female gametocytes staining, one for readout 2, specific female gametocytes staining). Error bars denote the standard deviation. The first curve is referred to killing effect and the second curve to retention effect normalized on DMSO negative control. Chemical structure of the compound is represented for each DRC. Hits and readouts have been selected among the ones with lowest variability, with the addition of TD-6450 and L-THP due to their high priority. Results did not differ too much between the two readouts, with the exception of hits MMV667494 and MMV030734 specifically actives on readout 2 (data not shown). A) DRCs of four selected hits from ReFrame library that showed a similar killing and retention effect. Readout 2 has been selected for all of them except APPCL. B) DRCs of four selected hits (three from ReFrame library and one from Pathogen Box) that showed higher killing than retention effect. Readout 2 used for all of them. C) DRCs of four selected hits (two from ReFrame library and two from Kinase Inhibitors Box) that showed higher retention than killing effect. Readout 2 used for all of them except TD-6450.

FIG. 5. Effects of TD-6450 on asexual parasites Synchronised rings exposed 48-hours to the compound, stained with Sybr-G and then quantified by FACS. NITD609 used as positive control and DMSO (not shown) used as negative control.

FIG. 6. Cumulative dot-plot of 5 experiments (effect after 24-hours drug exposure, on the left) and 4 experiments (sustainability of the effect 24-hours after washing out the compounds, on the right) where stage V gametocytes of 3D7 pULG8-GFP P. falciparum strain were exposed to fixed dose of TD-6450 and NITD609 given individually, and the combination of both. Each point shows the retention rate of a single well of a 96-well microsphiltration plate. DMSO is used as a negative control. Black line corresponds to median, with error bars indicating the interquartile range. One, two and three asterisks indicate that p-value are comprised between and 0.01, 0.01 and 0.001, and 0.001 and 0.0001, respectively; four asterisks indicate a p-value inferior to 0.0001.

### EXAMPLE

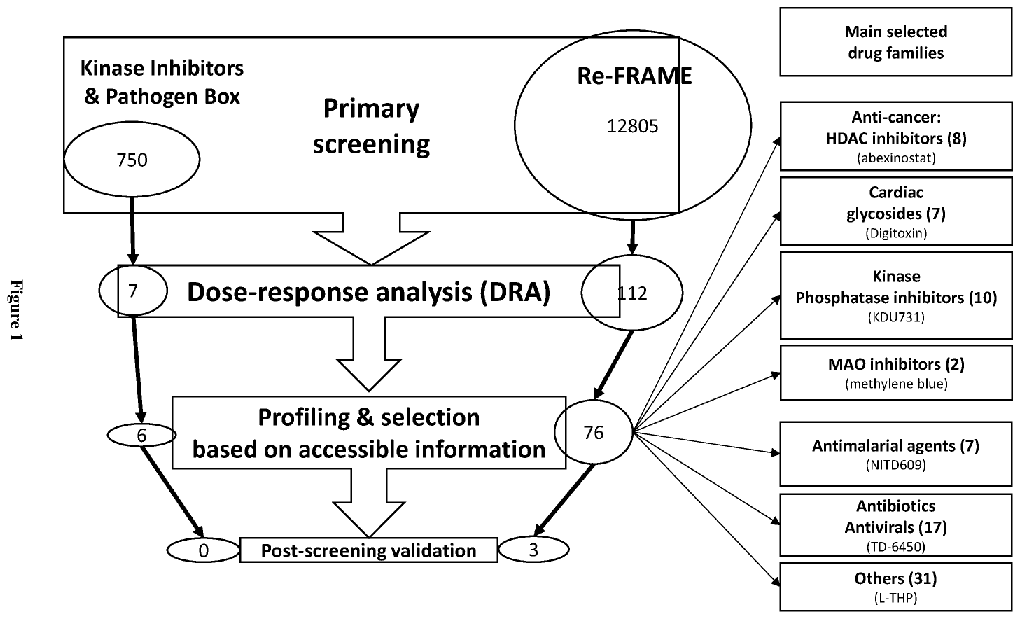

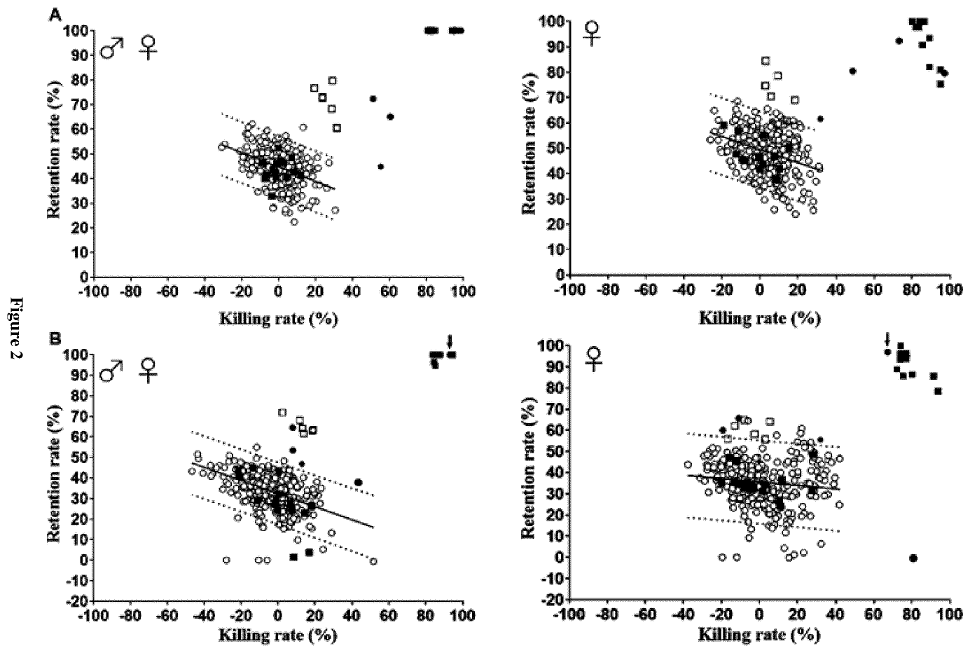

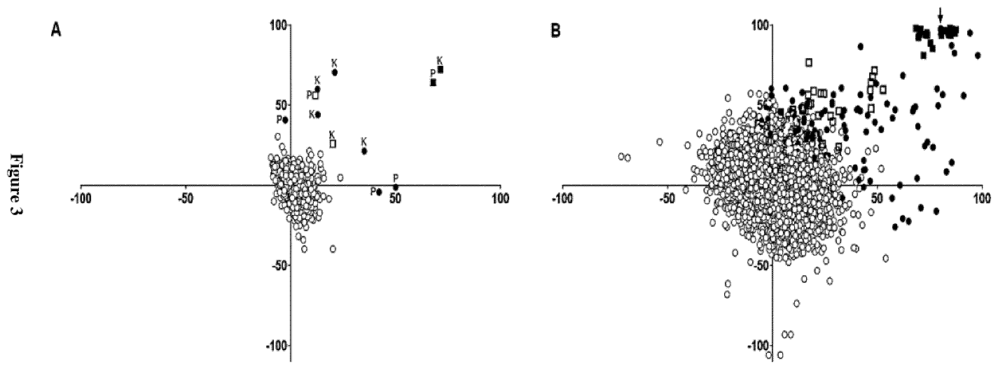

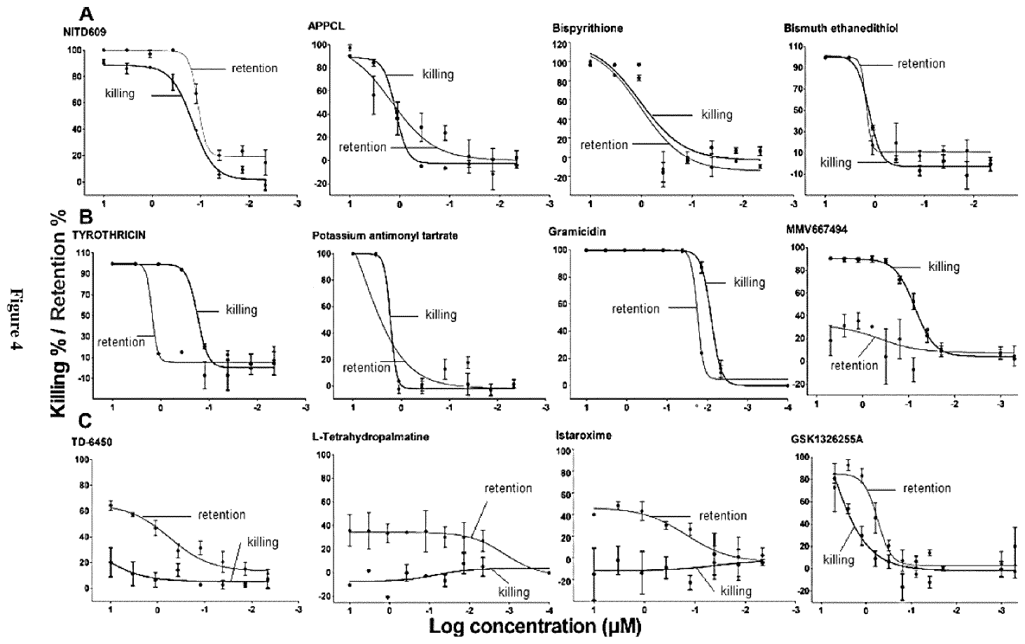

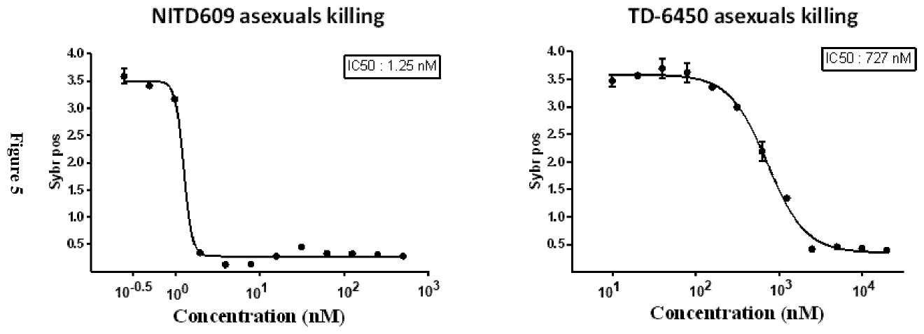

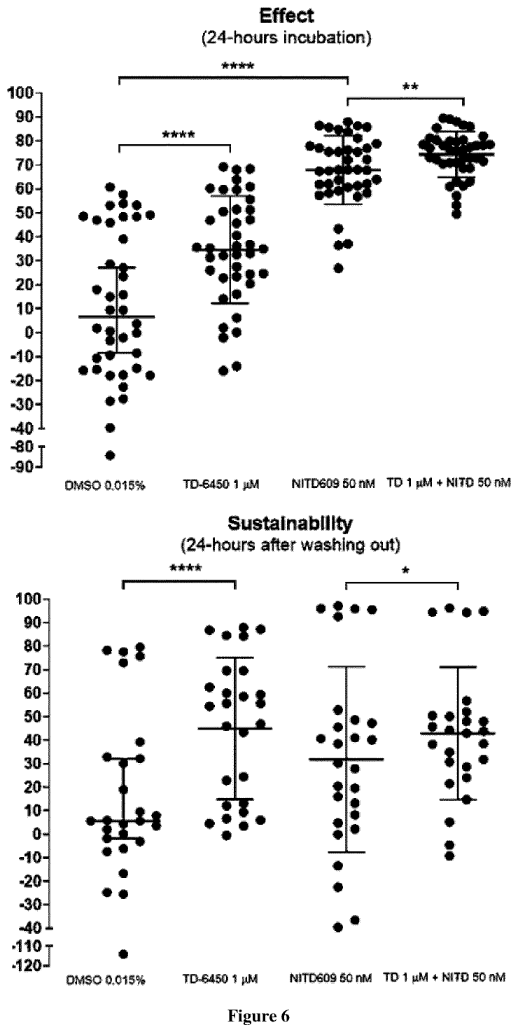

The spleen clears rigid erythrocytes from the circulation. Drug-induced stiffening of Plasmodium falciparum intra-erythrocytic sexual stages (mature gametocytes) is therefore expected to block the transmission of malaria. By screening 13 550 compounds with spleen-mimetic microfilters, we identified 82 compounds that stiffen mature gametocytes. Eight active families were identified, including known anti-malarial, antimicrobial or anticancer agents, amongst others. Hit prioritization based on accessible safety and pharmacokinetics data in humans identified 3 leading candidates (FIG. 1, FIG. 2A-B, FIG. 3A-B, FIG. 4A,B,C). NITD609 displayed killing and stiffening effects (IC50 of 100 and 50 nM, respectively), while TD-6450 and L-THP had a pure or predominant stiffening effect (IC50 of 600 and 5 nM, respectively)(FIG. 5). These values are lower than or close to peak plasma concentrations in humans. Stage V gametocytes of 3D7 pULG8-GFP P. falciparum strain were exposed to fixed dose of TD-6450 and NITD609 given individually, and the combination of both. Results are depicted in FIG. 6. Clinical trials with these strong malaria transmission-blocking candidates are now envisioned.

### REFERENCES

Throughout this application, various references describe the state of the art to which this invention pertains. The disclosures of these references are hereby incorporated by reference into the present disclosure.

1. A method of blocking transmission of a Plasmodium parasite by an infected subject comprising administering to the subject a therapeutically effective amount of a drug that increases rigidity of iRBCs selected from the group consisting of TD-6450, NITD609, and L-THP.

2. The method of claim 1 wherein the subject is infected by a Plasmodium parasite selected from the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium.

3. The method of claim 1 wherein the subject is infected by Plasmodium falciparum.

4. The method of claim 1 wherein the drug the that increases rigidity of iRBCs is TD-6450 and is administrated in combination with NITD609.

5. The method of claim 1 further comprising administering to the mammal at least one additional antimalarial compound.

6. The method of claim 5 wherein the at least one additional antimalarial compound include primaquine, bulaquine, artemisinin and derivatives thereof, chloroquine, hydroxychloroquine, mefloquine, amodiaquine, piperaquine, pyronaridine, atovaquone, tafenoquine, methylene blue, a trioxaquines, an endoperoxides, decoquinate, a 9-anilinoacridines, doxycycline, azithromycine, erythromycine, spiramycine, pyrimethamine, sulfadiazine, sulfamethoxazole, an HIV-protease inhibitors, and a natural products sueli as neem, epoxomicin, harmonine, and riboflavin.

7. The method of claim 5 wherein the drug is administered with a conventional curative treatment by a known anti-malarial compound at the beginning or at the end of a malaria attack.

8. The method of claim 6, wherein the endoperoxide is OZ 439 or OZ 277.

9. The method of claim 6, wherein the natural product is neem, epoxomicin, harmonine or riboflavin.
